(19) United States
(12) Patent Application Publication
(10) Pub. No.: US 2020/0258595 A1
(43) Pub. Date: Aug. 13, 2020

(54) METHODS OF FILTERING SEQUENCED MICROBIOME SAMPLES

(71) Applicant: International Business Machines Corporation, Armonk, NY (US)

(72) Inventors: Kristen L. Beck, San Jose, CA (US); Niina S. Haiminen, Valhalla, NY (US); Mark Kunitomi, San Francisco, CA (US); James H. Kaufman, San Jose, CA (US); Laxmi Parida, Mohegan Lake, NY (US); Matthew A. Davis, San Jose, CA (US)

(21) Appl. No.: 16/271,980

(22) Filed: Feb. 11, 2019

(51) Int. Cl. G16B 30/00; C12Q 1/6809; C12N 15/10

(52) U.S. Cl. CPC G16B 30/00; C12N 15/1003; C12Q 1/6809

(57) ABSTRACT

A method of electronically separating host and non-host sequence data (sequenced DNA, RNA, and/or proteins) utilizes electronic host filters, which can be generated on a just-in-time basis by a cloud-based software service. Host reads and non-host reads of a given sample are separated and stored in separate data repositories. Also disclosed is a cloud-based software service utilizing the method. The non-host reads resulting from the host filtration process can then be profiled more accurately for the microorganism content therein.

### BACKGROUND

The present invention relates to methods of filtering host nucleic acid and protein sequences from non-host sequences in a sample of a microbiome, and more specifically, to an online filtration service for removing host sequences.

When sequencing a microbiome, genetic information is collected from an environmental niche to describe that habitat/ecosystem. The microbial life resides in/on a host. The host can be known (e.g., in the event of a human microbiome) or unknown (e.g., in a mixed food ingredient). The sequence data from host content interfere with the identification of the microbes present. There are no systems in place to identify and filter such obstructive data when processing microbiome samples in a fully automatic fashion.

For many microbiome applications, it is important to separate or distinguish raw reads associated with microbial content of a sample with reads associated with the host content. Because the host and microbes can share common sequences, failure to separate host content before performing other analyses can lead, for example, to gross or subtle false positive identification of microbes. The host components can be genetic material (DNA or RNA sequencing reads) originating from a human host (in a clinical sample), other animal host, plant host, food host (i.e., plant and/or animal food components as in a food safety sample), or another host.

For microbiome applications, a large number of sequencing reads is required to accurately identify individual microbes because a very small fraction of the total reads comes from microbial content (compared to the host content). A reference database of all possible host content would be prohibitively large and require more single machine memory than is currently practicable. Filtering the possibly hundreds of millions of sequencing reads from the host using a large database would be slow or would require parallel processing using a large number of processors. This is currently not feasible, especially when time-to-result is critical in real applications (e.g., for food safety where decisions about downstream usage of material are made based on the microbiome profile).

Thus, a need exists for methods of rapidly identifying and removing host-related data from a sequenced sample of a microbiome.

### SUMMARY

Accordingly, a method is disclosed, comprising:

obtaining an initial set of sequence reads from a sample containing DNA, RNA, and/or proteins of multiple organisms, the initial set comprising i) a first subset of sequences corresponding to a first organism and ii) a second subset of sequences corresponding to a second organism;

identifying the first organism;

forming an electronic filter containing sequences of DNA, RNA, and/or proteins of the identified first organism; and

removing from the initial set any sequences of DNA, RNA, and/or proteins matching the sequences of DNA, RNA, and/or proteins of the electronic filter, thereby forming a set of removed sequences and a set of remaining sequences;

wherein

the set of remaining sequences is suitable for identifying the second organism.

Also disclosed is a computer program product, comprising a computer readable hardware storage device having a computer-readable program code stored therein, said program code configured to be executed by a processor of a computer system to implement a method comprising:

obtaining an initial set of sequence reads from a sample containing DNA, RNA, and/or proteins of multiple organisms, the initial set comprising i) a first subset of sequences corresponding to a first organism and ii) a second subset of sequences corresponding to a second organism;

identifying the first organism;

forming an electronic filter containing sequences of DNA, RNA, and/or proteins of the identified first organism; and

removing from the initial set any sequences of DNA, RNA, and/or proteins matching the sequences of DNA, RNA, and/or proteins of the electronic filter, thereby forming a set of removed sequences and a set of remaining sequences;

wherein

the set of remaining sequences is suitable for identifying the second organism.

Further disclosed is a system comprising one or more computer processor circuits configured and arranged to:

obtain an initial set of sequence reads from a sample containing DNA, RNA, and/or proteins of multiple organisms, the initial set comprising i) a first subset of sequences corresponding to a first organism and ii) a second subset of sequences corresponding to a second organism;

identify the first organism;

form an electronic filter containing sequences of DNA, RNA, and/or proteins of the identified first organism; and

remove from the initial set any sequences of DNA, RNA, and/or proteins matching the sequences of DNA, RNA, and/or proteins of the electronic filter, thereby forming a set of removed sequences and a set of remaining sequences;

wherein

the set of remaining sequences is suitable for identifying the second organism.

Further disclosed is a method, comprising:

inputting a sample into a sequencer, the sample including both a microbial target and a matrix on which the microbial target exists, the sequencer outputting reads of nucleic acids detected in the sample;

subjecting the reads to a quality control process to eliminate (i) part or all of those reads below a desired threshold and/or (ii) those reads originating from contaminants unrelated to the target or matrix;

determining the matrix identity by string matching a minority of the reads;

retrieving sequences related to the determined matrix by accessing a reference database;

in view of the retrieved sequences, dividing the reads into a first object store directed to the matrix and a second object store directed to the microbial target and any residuals, thus effectively filtering the host from the microbial target; and

using at least one of the object stores in a follow-on application.

The above-described and other features and advantages of the present invention will be appreciated and understood by those skilled in the art from the following detailed description, drawings, and appended claims.

### BRIEF DESCRIPTION OF THE SEVERAL VIEWS OF THE DRAWINGS

FIG. 1 is a flow diagram showing a preferred embodiment of the disclosed method to remove host sequences from sample reads.

FIG. 2 is a graph illustrating hypergeometric cumulative distribution values for fixed total size N=100,000,000 reads, sample size n=10,000 reads, and thresholds x=0.001 to 0.1. The x-axis represents observed read count k.

FIG. 3 is a block diagram showing a structure of a computer system and computer program code that may be used to implement the disclosed method

FIG. 4 is a diagram depicting a cloud computing environment capable of implementing a preferred embodiment of the disclosed method.

FIG. 5 depicts abstraction model layers according to an embodiment of the present invention.

### DETAILED DESCRIPTION

Methods are disclosed for automatically and programmatically filtering and removing host-related DNA, RNA, and protein sequence data from a sequenced sample (e.g., of a microbiome). The resulting remaining set of sequence data (the filtered set) can then be used to more accurately identify the organism(s) therein. The methods are adaptable to machine learning.

The term “host” as used hereinafter refers to any sequence data sought to be removed from (i.e., programmatically filtered from) a given sample of sequence data, regardless of any functional relationship, or lack thereof, between organisms of the sample. A functional relationship in the sense of providing nourishment or sustenance by an organism to another organism of the sample may or may not exist. The set of sequence data removed by the programmatic filtration process is referred to herein as “host” sequence data, host sequences, matrix, or set of removed sequences. The set of sequence data that remains after the filtration process is referred to herein as “non-host” sequence data, non-host sequences, non-matrix, filtered set, or set of remaining sequences.

No restriction is placed on the organisms from which the host sequences originate. The host sequences can be from one or more macroorganisms (e.g., birds, fish, reptiles, humans, other mammals), one or more microorganisms (e.g., bacteria, viruses, protozoans, fungi, parasites), or combinations thereof. Likewise, no restriction is placed on the organisms from which the non-host sequences originate. The non-host sequences can be those of one or more macroorganisms, one or more microorganisms, or combinations thereof. A macroorganism is an organism visible to the unaided eye (e.g., human, other mammals, fish, shell fish, birds, trees, food plants). A microorganism (microbe) is an organism not visible to the unaided eye (e.g., bacteria, viruses, microscopic fungi, protozoa).

As one example, it may be desirable to remove sequences of macroorganisms from a given sample in order to more accurately identify microorganisms of the given sample (e.g., a medical sample from a human digestive tract). In this instance, the host sequences are those of the macroorganism(s) of the sample (e.g., consumed animal and/or plant flood), and the non-host sequences are those of microorganisms of the sample (e.g., bacteria of the digestive tract). As another example, it may be desirable to remove sequence data of microorganisms from a given sample containing sequence data of a macroorganism (e.g., a decomposed forensic tissue sample) in order to make a more accurate identification of the macroorganism (e.g., identifying a specific person). In this instance, the host sequences are those of the microorganisms of the sample, and the non-host sequences are those of the macroorganism. In another example, it may be desirable to remove sequence data of one group of microorganisms (e.g., bacteria) from a given sample containing sequence data of another group of microorganisms (e.g., viruses). In this instance, the host sequences are those of the bacteria and the non-host sequences are those of the viruses. In another example, it may be desirable to remove sequence data of one group of macroorganisms (e.g., deer) from a given sample containing sequence data of another group of macroorganisms (e.g., ticks). In an embodiment, the sample of raw sequences outputted by a sequencer device includes a microbial target (non-host) and a matrix (host) on which the microbial target exists.

Also disclosed are a computer program, computer system, and online sequence filtration service (referred to herein as “filtration service” or simply “service”) for performing the disclosed methods. The computer program, computer system, and filtration service are preferably cloud-based. The customer uploads to the service a set of sequenced nucleic acids (the “customer set”) in the form of an electronic data stream. The sequences are in the form of text strings of varying length comprising nucleotide symbols (e.g., nucleotides A, G, T, C). The customer can upload a set of raw sequence data (“raw set”) directly from a high-throughput sequencer or upload a set of cleaned sequences (“clean set”) obtained after performing quality control (QC) on the raw set. In either case, the customer set includes host and non-host sequence data (sequenced DNA, RNA, and/or proteins).

For purposes of simplifying the following description, host sequences (removed sequences) will hereinafter be those of macroorganisms, and non-host sequences (remaining sequences) will hereinafter be those of microorganisms. Exemplary samples meeting these conditions include medical samples (e.g., clinical blood samples, digestive tract samples, other body fluid and tissue samples), food safety samples, soil samples, and water samples. It should be understood that the following description applies to the other above-described combinations of organisms.

The service can submit the received customer set (raw set or clean set) for host filtration as received. Preferably, the service performs quality control on the customer set, removing reads that fail quality control (referred to as “bad reads” or “failed reads”) and storing them in a bad read repository (also referred to as “failed read repository”) while streaming clean reads (referred to herein as the “initial set”) to a host identification microservice for identifying one or more host(s) organisms of the initial set. It should be understood that the initial set includes all clean reads of the customer set, and that the customer set and the initial set are handled by the service as data streams. Quality control can be performed during the upload of the customer set. Also, the host identification process can begin with clean reads of the initial set before the quality control process finishes. Preferably, the service is configured to operate as multiple microservices operating in parallel on data streams of the customer set and initial set. Where necessary, a data stream can be split into two or more data streams.

The service utilizes individual host filters (also referred to as electronic filters) to identify and remove host content of the initial set. A given host filter is a data table containing DNA, RNA, and/or protein data about a given host. Host filters can be built dynamically without a priori knowledge of host content of the initial set, as described in more detail further below.

A preliminary rapid identification of host(s) is possible by comparing the reads of the initial set to a small rRNA (e.g., prokaryotic 16S rRNA, eukaryotic 18S rRNA) single gene reference database of hosts and/or by comparing a subset of the initial set to a large reference database containing whole genomes of hosts. The reference databases used to identify a host in the initial set can be local or remote, internal to or external to the service. Preferably, these reference databases for determining host IDs are suitable for rapid identification of the host(s). In general, a host can be identified using between 0% and 20% of the sequence reads of the initial set, more specifically between 0% and 15% of the sequence reads of the initial set.

When a match to a host occurs, a host filter (electronic filter) is dynamically built by retrieving known DNA (including but not limited to whole genomes), RNA, and/or protein content of the matching host from: i) a host repository maintained by the service, ii) external reference databases (e.g., RefSeq at the National Center for Biotechnology (NCBI), the Sequence Read Archive (SRA) at NCBI) or iii) combinations of the foregoing. The host filter is then used to find and remove all matching host content (reads) from the initial set, thereby producing a set of removed sequences (host sequences) and a set of remaining sequences (non-host sequences or “filtered set”). The host sequences and non-host sequences are stored in a separate data stores.

The host sequences and/or the non-host sequences can be submitted to follow-on applications (e.g., pathogen tracking applications, applications for measuring and tracking antimicrobial resistance, and the like) and/or additional analyses. The customer can receive the filtered set, the removed host sequences, the results of any follow-on applications and/or additional analyses, a list of matching hosts, and the bad reads of the quality control process.

An existing host filter can be dynamically updated with any new content added to external reference databases since the most recent version of the host filter and/or by adding host content obtained from customer sets as they are processed.

The service can identify host content also by comparing the initial set with existing host filters maintained by the service. When a match to a host is found, the matching host filter can be updated by downloading known genetic data for the matching host from internal or externally maintained host reference databases. The updated host filter can then be used to filter the initial set, thereby removing sequence reads of the initial set matching the sequence data of the updated host filter.

Failure to find a host match to internal or external host reference databases indicates a new host (e.g., a previously undiscovered animal or thought to be extinct bird or fish species). In this instance, the service can build a new host filter from additional host-related data provided by the customer.

The method allows data processing to occur rapidly and automatically by streaming data directly from a device (i.e., high through-put sequencers) to a cloud service pipeline with provisions to limit downstream analyses (e.g., identification of organisms of the sample) until appropriate binning of data (e.g. separating host and non-host reads) has occurred. The method allows for more accurate profiling of the non-host sequences (e.g. microorganism(s)) of the sample.

### Definitions

The following definitions are applicable.

Abbreviations A, C, G, and T refer to nucleotide bases adenine, cytosine, guanine, and thymine, respectively.

“Base substitution” refers to certain bases of a sequence read being different from the corresponding bases on a reference genome when the sequence read is mapped to the reference genome.

The abbreviation “bp” means “base-pair” (e.g., a read of 100-bp means that one DNA read has 100 nucleotides in the polymer chain).

A “clade” is a group of biological taxa (such as species) that includes all descendants of one common ancestor.

A “contig” is a set of overlapping DNA sequences that together represent a consensus sequence of DNA or a region thereof.

A “consensus sequence” is the calculated order of the most frequent residues found at each position in a sequence alignment.

“Copy number” means the number of copies of a gene or plasmid within a genome. The copy number can vary from individual to individual.

“Coverage” or “depth of coverage” is the number of times a given sequence from a genome is represented in the set of sequences derived from that genome.

“Deletion” is a term used to describe missing bases in a sequence read compared with a reference genome.

“DNA” is deoxyribonucleic acid.

A “gene” is the basic unit of heredity, a linear sequence of nucleotides along a segment of DNA that provides the coded instructions for synthesis of RNA, which, when translated into protein, leads to the expression of a hereditary trait.

“Genetic distance” is a quantitative measure of the divergence of one or more regions of DNA and/or RNA between species or populations of species. Genetic distance can be based on whole genome-whole genome distances, gene-gene distances, protein domain-protein domain distances (i.e., the portions of the DNA encoding for a particular protein domain), protein-protein distances (i.e., the portions of the DNA encoding for a whole protein), or protein domain-protein domain distances based on an amino acid distance metric. More specifically, genetic distance is a measure of the differences in nucleotide sequences of the k-mers with respect to whole genomes, genes, and/or other genetic regions of interest. Thus, the average number of codon or nucleotide differences per gene can be a measure of genetic distance. For the present work, the genetic distance is a numeric distance calculated between each pair of genomes of the reference database using MASH (which utilizes the MinHash algorithm). The MinHash algorithm calculates distance from a Jaccard index. The Jaccard index is calculated from “sketches” of the k-mers, which are diagrams showing the similarity and differences between k-mers of the pair of genomes.

A “genome” is the total genetic content of a microorganism. In the case of bacteria, the genome is DNA.

A “ground truth dataset” is a dataset formed by direct observation (measured data) as opposed to data obtained by inference or assumption.

Herein “high-throughput sequencing” (HTS) is any method of sequencing a nucleic acid that is highly parallel and does not involve cloning the nucleic acid. A genome or metagenome is cut into a large number of fragments, and the fragments are sequenced in parallel.

“Homology” refers to the similarity of sequences (e.g., DNA, RNA, Protein, etc.) arising from a common ancestry.

“Hybridization” is the formation of double-stranded helix from single-stranded complimentary pairs of DNA and/or RNA by annealing.

“Insertion” is a term used to describe additional bases in a sequence read compared with a reference genome.

“INDEL” is a term used to describe an insertion or deletion in a read when trying to find the best alignment of the read to a reference genome.

The term “k-mer” means a sub-sequence of a read obtained through DNA sequencing having k number of nucleotide base units, where k is a positive whole number greater than 1.

Herein, a “database” is an electronic file for storing and retrieving data. Databases are also referred to herein as data tables. Data tables comprise rows and columns (i.e., fields) of data. The rows are formally called tuples or records. A data table comprises one or more records, each record comprising one or more defined fields having respective defined data types (e.g., text, numeric, date, time, memo, and so on) and defined field lengths where applicable. A working data table comprises at least one record containing data in one or more fields of the record. The data tables are located on data storage devices, which can be remote or local relative to the user input/output devices. A “database system” comprises at least one data table and a database management software program for managing the storage and retrieval of data to and from the data tables. The database management programs can be remote or local relative to the data tables and/or the end user. A Relational Database Management System (RDBMS) is a database management system (DBMS) that uses relational techniques for storing and retrieving data using data tables. A relational database system can have many data tables, and each data table can have multiple records and multiple fields within each record. A data table in a relational database system can be accessed using an index. An index is an ordered set of references (e.g., pointers) to the records or rows in a data table. The index is used to access each record in the file using a key (e.g., one or more of the fields of the record or attributes of the row). Without an index, finding information in a large data table would require a resource-intensive time-consuming scan (e.g., linearly) of each record of a table. Indexes provide a faster alternate technique of accessing data contained in one or more data tables that are linked by a common key. Users can create indexes on a table after the table is built. An index is based on one or more columns (fields) of a given table.

A “k-mer database” is a database in which a given record comprises a field for storing a k-mer of a nucleic acid sequence of one or more organisms. Another field of the record stores a taxonomic ID that associates the k-mer to a lowest common ancestor node (LCA) of a taxonomic tree. As will be described below in more detail, other fields of the record can store reference IDs to a reference taxonomy. Still other fields of the record can store metadata associated with the k-mer and/or the nucleic acid sequence from which the k-mer originated.

Kraken is a taxonomic classifier that assigns taxonomic labels to DNA sequences, including k-mers. Kraken uses k-mers from a sequence read to query a reference database containing k-mers from reference genomes (i.e., the genomes of RefSeq Complete at NCBI) for matches. The k-mers are mapped to the lowest common ancestor (LCA) of all genomes known to contain a given k-mer. Typically, the k value for a k-mer query is 31 but this value can be modified by the user. For typical queries, k can be a positive whole number in the range of about 10 to about 1000.

Herein, a “Kraken database” is an electronic file containing k-mers assigned to a taxonomic hierarchy by the Kraken classifier.

A “locus” (plural loci) is a position on a genome (e.g., gene, regulatory element, origin of replication).

“Mapping” a sequence read is a process of finding the position or coordinate of a sequence read on the reference genome.

A “metagenome” is all the genetic information of a sample.

“Metagenomics” is the analysis or study of metagenomes.

A “metatranscriptome” is the collection of all RNA transcripts of a sample.

“Metatranscriptomics” is the analysis or study of metatranscriptomes.

A “microbiome” is a community of microorganisms that inhabit a particular environment (e.g., microbes of the human gut), or a sample taken therefrom.

“Microservice” architecture is an architectural style of computer software applications written as a suite of independently deployable, small, modular services. Each microservice runs a unique process and communicates through a well-defined, lightweight mechanism to serve a business goal.

“Miscalling” refers to a sequencing error where a nucleotide in a sequence read is different from the true nucleotide.

“Origin of replication” is the locus at which DNA replication begins.

Operational taxonomic units (OTUs) are used by taxonomy classifier systems (e.g., Kraken classifier) to categorize the k-mers based on sequence similarity. For example, in 16S rRNA metagenomics, OTUs are clusters of similar sequence variants of the bacterial 16S rRNA marker gene sequence. Each cluster represents a taxonomic unit of a bacterial species or genus depending on the sequence similarity threshold. Typically, OTU clusters are defined by a 97% identity threshold of the 16S gene sequences to distinguish bacteria at the genus level. Species separation requires a higher threshold of 98% or 99% sequence identity, or the use of exact sequence variants instead of OTU cluster.

A “perfect match prefix” is a k-mer of a sequence read that is identical to, or a perfect match to, some equal-length k-mer(s) of the reference genome. The k-mer of the sequence read is used to initially anchor the sequence read on the reference genome.

A “plasmid” is a self-replicating extrachromosomal circular DNA that replicates independently of the bacterial chromosome and carries genes for functions not essential for growth.

A protein is a polymer of amino acids joined together by peptide bonds.

A “protein domain” is a region of a given protein sequence that may have independent functional or structural features from the rest of the protein chain. A protein domain can have a particular shape.

A “proteome” is the complete set of proteins that is or can be produced in a cell, tissue, organism, or environment.

“Proteomics” is the analysis or study of proteomes.

A “quality value” is an assigned value given to each nucleotide in a sequence read that reflects the likelihood of miscalling the nucleotide. The higher the quality value is, the lower the likelihood of miscalling.

A “reference genome” is a genome from the same species or close species that has already been sequenced.

“RNA” is ribonucleic acid.

“mRNA” refers to messenger RNA. The mRNA codes for amino acid sequences composing proteins.

“rRNA” refers to ribosomal RNA.

“tRNA” refers to transfer RNA. A tRNA transports a specific amino acid to a ribosome for synthesis of a protein.

An “RNA transcript” is an RNA produced through the process of transcription of DNA.

“Sample” means any sample containing DNA and/or RNA capable of undergoing analysis using the disclosed methods.

“Sequencing” refers to a process of determining the precise order of base residues (i.e., nucleotides) in a nucleic acid (e.g., DNA, RNA).

A “sequence” is a fragment of a nucleic acid (e.g., RNA, DNA) that has been sequenced (i.e., the order of the nucleotides bases is known).

A “sequence read” or “read” is a finite length or fragment of a nucleic acid that is output by a sequencing instrument. For example, a read from an Illumina sequencer is 100-150 base pairs in length today. Sequencing may also be done on “paired end” reads where two reads are connected by a spacer (that is not read), increasing the effective read length to 300 or more and covering a larger region of the genome.

A “sequence alignment” is a way of arranging sequences to identify regions of similarity, which may be a consequence of functional, structural, or evolutionary relationships between the sequences.

“Shotgun sequencing” is a quasi-random process in which a nucleic acid is broken up into many random smaller fragments that are individually sequenced. The sequences are ordered based on overlapping regions of genetic code and reassembled into the complete sequence of the nucleic acid.

“Taxonomy” is a biological scheme of classification of organisms. Herein, for bacteria, the hierarchy is domain, kingdom, division, phylum, class, order, family, genus, species, sub-species, and strain. Each of the foregoing classifications is a “rank” on the taxonomic tree.

A “taxonomic tree” herein is a data structure for classifying organisms. The taxonomic tree comprises nodes (i.e., taxa, singular taxon) that are grouped into “parent nodes” linked to “child nodes”. Parent nodes are depicted above child nodes in a tree diagram. Child nodes are taxonomic descendants of parent nodes. For example, a genus (parent node) can be linked to two or more species (child nodes). The taxonomic tree can be rooted (i.e., known ancestral root) or unrooted (i.e., unknown ancestral root), bifurcating (i.e., two child nodes per parent node) or multi-furcating (i.e., more than two child nodes per parent node). Typically, the taxonomic tree is in the form of a “binary tree” (i.e., each parent node has two child nodes). A “leaf node” is a child node having no descendants (e.g., the species of a genus). In the self-consistent taxonomy, each leaf node has one genome. “Internal nodes” are all nodes other than the leaf nodes.

“Transcription” is the process of forming an RNA from a DNA template.

### Preferred Embodiment

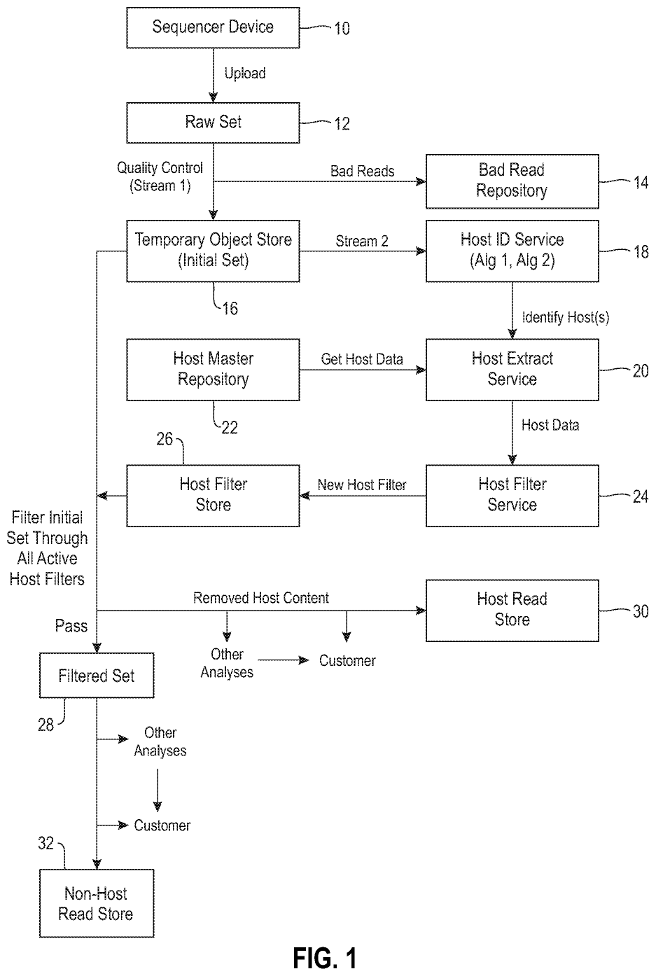

FIG. 1 is a flow diagram showing a preferred embodiment of the disclosed method. The method begins with the customer uploading to an online service (preferably cloud service) a set of raw sequence reads (Raw Set 12), preferably directly from a DNA/RNA sequencer device 10. The reads as Stream 1 pass through quality control (QC) for trimming and removal of common contaminants (e.g., PhiX filtering with standard available tools (Trimmomatic, TrimGalore, etc.). Reads that fail quality control (QC) are discarded or stored in a Bad Read Repository 14 (also referred to as Failed Read Repository). Reads that pass QC are the initial set for host filtration and are moved to a temporary object store 16. The initial set comprises a first subset of sequences (host sequences) corresponding to a first organism (host organism) and ii) a second subset of sequences (non-host sequences) corresponding to a second organism (non-host organism).

From the temporary object store 16, sequence reads of the initial set are passed in Stream 2 to the Host ID Service 18. The purpose of the Host ID Service is to rapidly identify host components of the initial set that should be removed (filtered). The Host ID Service can perform quantitative or non-quantitative host profiling as separate services. The rapid host identification process can be based, for example, on a lightweight single gene database such as eukaryotic 18S rRNA gene database and/or prokaryotic 16S rRNA, an internal transcribed spacer (ITS) gene plant database, a cytochrome C oxidase subunit I (COI) gene animal database, a core eukaryotic gene database, a complete eukaryotic genome database, and/or a more comprehensive distributed sequence database. Mapping reads to host organism(s) in the database can be achieved with tools such as, for example, Kraken (for exact k-mer based rapid classification), Bowtie 2 (a standard read mapper), BLAST (for sensitive genome aligning), and the like. The gene databases and profiling tools can be used singularly or in combination. Identification of the host organism can be accomplished using a minority of reads of the initial set, typically 20% or less of the sequence reads of the initial set, preferably between 0% and 15% of the sequence reads of the initial set.

The Host ID Service can use an algorithm designated Alg 1 to determine when the evidence for a host component having a given ID is in the sample. Alg 1 can do the identification by matching one ID (one host component) at a time based on evidence in the stream of data, or it can identify sets of IDs based on data in the stream in some time interval Δt. In one possible variation of Alg 1, a given host ID can be declared present when the number of reads assigned to the given host ID exceeds a pre-assigned threshold percentage of total reads processed at any set point in the stream.

Another possible variation of Alg 1 is based on the following relationship. Given a sub-sample of reads of size Ns, let m_{id}=g_{id}, Ns), where m_{id} is the minimum number of reads required to trigger a program call that host component having taxonomic ID=“id” is present. The function f depends on r, g_{id} and Nₛ, where r is the average read length, and g_{id} is the host taxonomic ID in the host reference database.

When evidence of a host of a given ID exceeds the required threshold, a trigger event is sent to a Host Extract Service 20 indicating a host match. The trigger event contains the ID or set of IDs found.

The Host ID Service uses a second algorithm Alg 2 to determine when all required host IDs have been found and host ID filtration can stop. Alg 2 also determines when the creation of new host filters can stop (below).

Stopping can be automatic, for example, when all reads have been processed and the stream is closed for a given sample. Stopping can also be based on a pre-determined number of reads of the sample Nₛ (a subset of the total reads N) when all host IDs with a relative abundance over a pre-determined threshold x have been discovered with high probability (such as Pr>0.999). The size of the subset Nₛ and probability Pr can be computed according to Equation (1) further below based on the total number of reads N and the desired abundance threshold x, where x is a number in the range of 0 to 1.

Another stopping point for Alg 2 can be based on a minimum number of reads m_{g} that do not match a given host g. Assume average read length r and host g in the host database. Given a sub-sample of size Ns, let m_{g}=f(r,g,Nₛ) where f is a function that gives the minimum number of reads that must be present so that host g is called. Alg 2 stops with respect to host g when for no g the number of reads exceeds m_{g}.

Another stopping point for Alg 2 can be based on the relative abundance of new host components. For example, Alg 2 can stop when the relative abundance of new host components is less than the fraction of non-host components by a pre-defined fraction f.

Returning to FIG. 1, when the Host Extract Service 20 is notified that one or more hosts were found, genetic host data are extracted from a Host Master Repository 22 maintained by the service. Host data can also be obtained from external database sources. The host data include the genomes, genes, k-mers, or k-mer databases associated with the host ID(s). This relevant host genomic data are then sent by the Host Extract Service 20 to the Host Filter Service 24, which creates a new host filter using the extracted host data. An “ID complete” notification can be sent by the Host Extract Service to signify a stop in Alg 2. The new host filter is then sent to the Host Filter Store 26.

The initial set of sequence data in the Temporary Object Store 16 is then passed through the new host filter and any existing host filters in the Host Filter Store 26, producing filtered set 28. Reads of the initial set that match reads of the host filters are removed (filtered) from the initial set and sent to a Host Read Store 30. The resulting filtered set 28 is moved to a Non-Host Read Store 32.

The removed host reads and/or non-host reads (i.e., filtered set 28) can be submitted to additional analyses when filtering is complete. The non-host reads, host reads, and the results of additional analyses can then be returned to the customer in the form of separate electronic files with accompanying reports.

The new host filter joins a set of active host filters that grows over time. Reads in the active stream (i.e., the initial set) pass through all active host filters. Existing reads in the non-host read store, which are already filtered, are re-filtered only through the new host filter.

The removed host reads can be combined with an existing host filter to generate a new host filter (not shown).

The purpose of the complete set of filters is to separate reads into two separate storage systems or object stores. Reads identified with any host ID are moved to the sample host object store (indexed by sample ID). Reads not associated with any host ID are moved to the non-host object store (indexed by sample ID).

### Equation 1

Assume N total reads, n sampled reads, a threshold x for minimum relative fraction of organism O reads within total reads (where 0≤x≤1), and a threshold for minimum number of reads m_{id} required to ID the organism O. Then the probability Pr that at least m_{id} reads from organism O are detected is:

$$P r \geq 1 - {\sum }_{0 < = k < {m}_{i d}} P ( X = k ) , \qquad ( 1 )$$

where P is the probability mass function of the hypergeometric distribution (with parameters N objects, K=N times x desirable objects, n draws, k successful draws). A random variable X follows the hypergeometric distribution if its probability mass function (pmf) is given by:

$$P ( X = k ) = \frac{( \begin{matrix}K \\ k\end{matrix} ) ( \begin{matrix}N - K \\ n - k\end{matrix} )}{( \begin{matrix}N \\ n\end{matrix} )} , \qquad ( 2 )$$

where

N is the total number of reads in the sample,

K is the number of reads for organism O calculated in the population N,

n is the number of sampled reads,

k is the number of observed reads for organism O in the number of sampled reads,

$$( \begin{matrix}a \\ b\end{matrix} )$$

is a binomial coefficient.

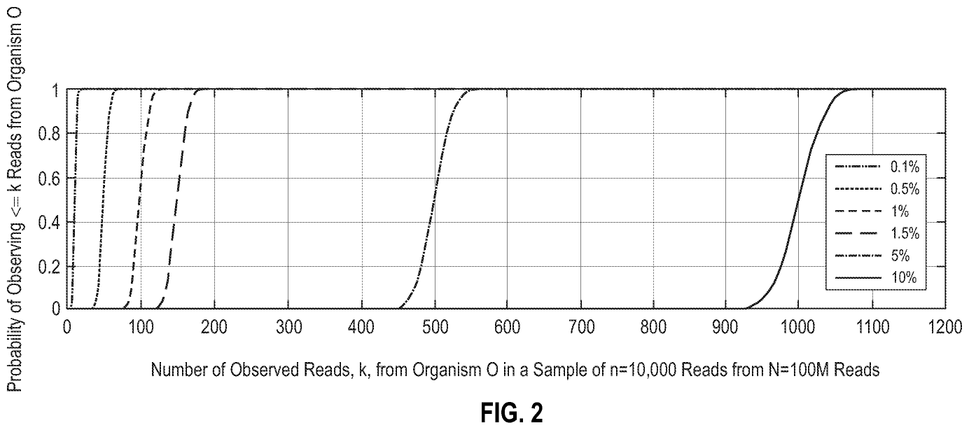

FIG. 2 (graph) illustrates of hypergeometric cumulative distribution values for fixed total size N, sample size n, and thresholds x=0.001 to 0.1. The x-axis represents observed read count k.

### Summary of the Host Filtration Service

Preferably, the host filtration service is configured as a cloud based software service where DNA, RNA, and/or protein data (reads) are uploaded to the service as a stream directly from a measurement device such as a high throughput sequencer. The system is configured with multiple microservices providing different and parallel processing functions on multiple computer nodes. Processing of the reads in one or more microservices can begin before the upload to quality control is complete. For example, the quality control microservice can send clean reads as a stream to the Host ID microservice while the upload from the measurement device continues. Bad reads from the quality control process are removed from the main stream and discarded or preferably stored separately. The emerging stream from a microservice can be split into one or more streams feeding other microservices (e.g., the emerging stream from the quality control microservice can be split into streams feeding the host ID microservice, the Bad Read Repository, and the Temporary Read Store while quality control is conducted). Any microservice of the system can stream data to one or more microservices until receiving a stop notification, and in most instances a microservice can initiate its process on an incoming stream before other microservices complete their respective processes. For example, a branch of the streamed data emerging from quality control can be analyzed for matching host(s) by the Host ID microservice while the upload to the quality control microservice and the quality control process continue. The Host ID microservice can begin analyzing the incoming stream without prior knowledge of the host or host content. Concurrently, another branch of the streamed data emerging from quality control can move to a temporary read store awaiting host filtration.

Once a host ID has been made, the Host Filter microservice can begin its process of constructing a new host filter even if the upload to quality control has not finished. Similarly, reads stored in the temporary read store can begin filtration through any existing host filters maintained by the service while a new host filter is generated by the Host Filter microservice. Once a new host filter is complete, non-host reads of the sample that have been filtered through existing host filters are filtered again through a new host filter. Similarly, all other non-host reads stored in the Non-Host repository are filtered again through new host filters. If more than one host filter is generated by the Host Filter microservice, all filtered non-host reads of the sample and of the Non-Host Read repository are filtered again through each successive new host filter. Host reads removed by the filtration process are stored in the Host Read repository. All non-host reads passing the filtration process are stored in the Non-Host Read repository.

Each microservice can send notifications, including process complete notifications or stop notifications, in real time to other microservices operating in parallel. For example, the Host ID microservice can send notifications (including host component IDs) in real time to the Host Filter microservice as new host components of the sample are identified. As another example, the Host Filter microservice can dynamically launch new microservice filters in real time based on notification of new host content by the Host ID microservice. As yet other examples, the Host Filter microservice can selectively re-filter the non-host data through new filters as they are launched, and the Host Filter microservice can stop creating new filters upon notification from another microservice. Notifications can be sent by push or by pull.

The Host Filter microservice utilizes host and non-host data stores that can be created dynamically from available sequence data (nucleic acid and protein) of one or more organisms. Host filters can be generated on demand from a library of genomes, k-mers, precomputed k-mer (or Kraken) databases, genes, proteins, and/or protein domains.

From nucleic acid data sets, the service can generate or augment databases using a library of organism genomes, genes, eukaryotic indicator genes, organism protein domains, and/or organism k-mers. From protein data sets, host databases can be generated or augmented using a library of organism in silico translated genomes, organism proteins, conserved eukaryotic indicator proteins, organism protein domains, and/or organism k-mers. The service preferably bins data by organism or chosen rank within the organism's lineage. Other data can be binned by labels or metadata (e.g., a biomarker, pathogen, a controlled agent). The service can maintain separate data stores defined by category, including but not limited to host component data, non-host component data, food matrix component data, microbial data, virus data, fungi data, and microbiome data. DNA, RNA, or protein data sets are streamed to and from the separate data stores by the microservices. The data stores defined by category can be locked from downstream analysis services until receiving a “Host ID process complete” notification.

### Samples

A sample is obtained by the customer for sequencing. The sample can contain nucleic acids and proteins of one or more hosts and non-hosts (e.g., a human gut sample containing human DNA/RNA (host), microbial DNA (non-host), and consumed plant DNA/RNA (host). The nucleic acids can be of eukaryotic and/or prokaryotic in origin. Non-limiting examples of samples include water samples obtained from tap water, lakes, streams, field runoff, and sewage; swabbed samples from contact surfaces (e.g., building surfaces, countertops, furniture, utensils, clinical instruments, computer hardware, cell phones, door handles, doors, windows, screens, cabinets, cabinet doors, sinks, faucet); animal samples (e.g., blood, blood plasma, serum, cells, a cellular extract, a cellular aspirate, expectorant, sputum, saliva, mucous, urine, sweat, tears); and samples obtained from food, food-handling equipment, and surfaces contacted by food. The samples can be a solid or liquid containing water or no water.

The consumer prepares and submits the sample for high through-put sequencing by a sequencer device that is preferably in electronic communication with the cloud filtration service. The raw sequences (raw set) produced by the device are automatically sent as a text stream to the filtration service.

### Microorganisms

The samples generally contain microorganisms. Microorganisms include bacteria, fungi, viruses, protozoans, parasites, and combinations thereof.

Exemplary non-limiting bacterial species include Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus Thuringiensis, Bacteroides fragilis, Bacteroides gingivalis, Bacteroides melaninogenicus (also known as Prevotella melaninogenica), Bartonella henselae, Bartonella quintana, Bordetella, Bordetella bronchiseptica, Bordetella pertussis, Borrelia afzelii, Borrelia burgdorferi, Borrelia garinii, Borrelia recurrentis, Brucella abortus, Brucella canis, Brucella melitensis, Brucella suis, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydophila pneumoniae (previously called Chlamydia pneumoniae), Chlamydophila psittaci (previously called Chlamydia psittaci), Chlamydia trachomatis, Clostridium botulinum, Clostridium difficile, Clostridium perfringens (previously called Clostridium welchii), Clostridium tetani, Corynebacterium diphtheriae, Corynebacterium fusiforme, Coxiella burnetii, Ehrlichia canis, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Leptospira interrogans, Leptospira santarosai, Leptospira weilii, Leptospira noguchii, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycobacterium ulcerans, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Peptostreptococcus, Porphyromonas gingivalis, Prevotella melaninogenica (previously called Bacteroides melaninogenicus), Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Shigella sonnei, Spirillum volutans, Streptococcus agalactiae, Staphylococcus aureus, Staphylococcus epidermidis, Staphylococcus saprophyticus, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Streptococcus viridans, Treponema pallidum, Treponema denticola, Ureaplasma urealyticum, Vibrio cholerae, Vibrio comma, Vibrio parahaemolyticus, Vibrio vulnificus, Yersinia enterocolitica, Yersinia pestis, Yersinia pseudotuberculosis,

Non-limiting exemplary viruses include Adenovirus, Herpes simplex, type 1, Herpes simplex, type 2, Varicella-zoster virus, Epstein-barr virus, Human cytomegalovirus, Human herpesvirus, type 8, Human papillomavirus, BK virus, JC virus, Smallpox, Hepatitis B virus, Parvovirus B19, Human astrovirus, Norwalk virus, coxsackievirus, hepatitis A virus, poliovirus, rhinovirus, Hepatitis C virus, yellow fever virus, dengue virus, West Nile virus, TBE virus, Rubella virus, Hepatitis E virus, Human immunodeficiency virus (HIV), Influenza virus, Lassa virus, Crimean-Congo hemorrhagic fever virus, Hantaan virus, Ebola virus, Marburg virus, Measles virus, Mumps virus, Parainfluenza virus, Respiratory syncytial virus, Rabies virus, Rotavirus, Orbivirus, Coltivirus, Banna virus, and zika virus.

Non-limiting exemplary fungi include Candida albicans, Aspergillus fumigatus, Aspergillus flavus, Aspergillus clavatus, Cryptococcus neoformans, Cryptococcus laurentii, Cryptococcus albidus, Cryptococcus gattii, Histoplasma capsulatum, Pneumocystis jirovecii, Pneumocystis carinii, and Stachybotrys chartarurn.

Non-limiting exemplary protozoa include Entamoeba histolytica, Entamoeba coli, Entamoeba dispar, Entamoeba moshkovskii, Entamoeba angladeshi, Entamoeba hartmanni, Dientamoeba fragilis, Endolimax nana, lodarnoeba butschlii, Plasmodium malariae, Plasmodium falciparum, Plasmodium vivax, Plasmodium ovale, Naegleria fowleri, Acanthamoeba species, Balamuthia mandrillaris, Sappinia diploidea, Giardia larnblia, Giardia intestinalis, Giardia duodenalis, Toxoplasma gondii, Nippostrongylus brasiliensis, Cryptosporidium parvum, Cryptosporidium hominis, Cryptosporidium cams, Cryptosporidium felis, Cryptosporidium meleagridis, Cryptosporidium muris, Trichomonas vaginalis, Trypanosoma cruzi, Leishmania major, Leishmania tropica, Leishmania barziliensis, Leishmania mexicana, Leishmania guyanesis, Leishmania panamensis, and Trypanosoma brucei.

### Sequencing

Non-limiting methods of DNA/RNA sequencing include massively parallel signature sequencing (or MPSS), Polony sequencing, Roche 454 pyrosequencing method, Illumina (Solexa) sequencing, SOLiD sequencing, ion semiconductor sequencing, DNA nanoball sequencing, heliscope sequencing, single molecule real time sequencing (SMRT sequencing), solid state nanopore sequencing, protein based nanopore sequencing, sequencing by electrical tunneling currents, sequencing by host-assisted laser desorption ionization time-of-flight mass spectrometry (MALDI-TOF MS), microfluidic Sanger sequencing, transmission electron microscopy DNA sequencing, RNA polymerase (RNAP) sequencing method, in vitro virus high throughput sequencing (IVV-HiTSeq), and sequencing by hybridization. Multiple, fragmented sequence reads must be assembled together on the basis of their overlapping areas.

The foregoing methods can be used singularly or in combination. The sequencing methods can be applied to genome sequencing, genome resequencing, transcriptome profiling (RNA-Seq), DNA-protein interactions (ChIP-sequencing), and epigenome characterization. Preferably, the sequencing method(s) operates in a parallel mode (characterizing many sequences concurrently).

### Quality Control (QC)

Optionally, quality control of the raw sequences can be performed by purging sequences of poor quality, removing contaminating sequences introduced by the sequence methodology, and/or removing (trimming) sequences of low complexity. Sequences of low complexity include those containing contiguous repeating pairs of two nucleotides (e.g., . . . (GA)ₙ . . . where n is a whole number greater than about 3). Contaminating DNA/RNA sequences can originate from inadvertent human, other animal, and/or plant contact with the working sample. Non-limiting algorithms and software programs for trimming and cleanup of raw sequences include SolexaQA DynamicTrim, FASTX-ToolKit, ConDeTri, NGS QC Toolkit, FASTQC, and Trimmamatic. Preferably, quality control is performed by FASTQC and/or Trimmamatic, thereby forming a set of “clean” sequences of the control sample.

Optionally, the raw sequences (or the clean sequences) can be assembled by software to form what is referred to herein as raw contigs or clean contigs, respectively (not shown).

### Profiling the Filtered Set

Profiling the filtered set of reads (non-host sequences) can be performed as a downstream microservice of the cloud based service or by the customer.

Optionally, the sequences of the filtered set are assembled to contigs. K-mers of the sequences/contigs can then be mapped to reference genomes of a reference database using Burrows-Wheeler transformation based method or a similar technique.

The filtered set can be passed to publicly available intermediary programs such as BLAST for aligning k-mers of the sequences of the sample to the k-mers of the reference database, thereby identifying which organisms (e.g., microorganisms) are most likely to be present in the filtered set. Optionally, the intermediary program can conduct alignment of sequence data of the filtered set to raw sequences, contigs, and/or whole genomes of reference databases in order to increase the specificity of the organism identification. The intermediary program can perform a simple database search on a sequence, or alternatively, conduct pairwise sequence alignments, multiple sequence alignments, and/or pairwise genome alignments.

Other non-limiting software programs for aligning metagenomic and metatranscriptomic sequences include FASTA (simple search), ALLALIGN (pairwise, multiple alignments), BLASTZ (pairwise), DNASTAR (pairwise, multiple), AVID (pairwise genome), GMAP (genome alignment), and MGA (multiple genome alignment).

### Computer Hardware and Software

The computer system for implementing the present invention can take the form of an entirely hardware embodiment, an entirely software embodiment (including firmware, resident software, microcode, etc.), or a combination of software and hardware that may all generally be referred to herein as a “circuit,” “module,” or “system.”

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

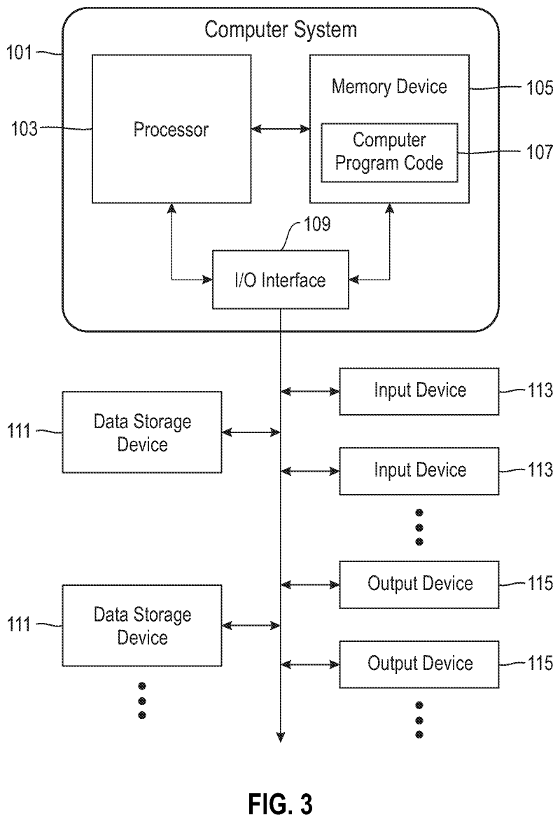

FIG. 3 shows an example of a structure of a computer system and computer program code that can be used to automatically and programmatically implement the processes of the invention, including filtering and removing host-related DNA, RNA, and protein sequences from a sequenced sample of a microbiome. In FIG. 3, computer system 101 comprises a processor 103 coupled through one or more I/O Interfaces 109 to one or more hardware data storage devices 111 and one or more I/O devices 113 and 115. Hardware data storage devices 111 can contain, for example, reference genome databases, host sequences, non-host sequences, and host filters.

Hardware data storage devices 111 may include, but are not limited to, magnetic tape drives, fixed or removable hard disks, optical discs, storage-equipped mobile devices, and solid-state random-access or read-only storage devices. I/O devices may comprise, but are not limited to: input devices 113, such as keyboards, scanners, handheld telecommunications devices, touch-sensitive displays, tablets, biometric readers, joysticks, trackballs, or computer mice; and output devices 115, which may comprise, but are not limited to printers, plotters, tablets, mobile telephones, displays, or sound-producing devices. Data storage devices 111, input devices 113, and output devices 115 may be located either locally or at remote sites from which they are connected to I/O Interface 109 through a network interface.

Processor 103 may also be connected to one or more memory devices 105, which may include, but are not limited to, Dynamic RAM (DRAM), Static RAM (SRAM), Programmable Read-Only Memory (PROM), Field-Programmable Gate Arrays (FPGA), Secure Digital memory cards, SIM cards, or other types of memory devices.

At least one memory device 105 contains stored computer program code 107, which is a computer program that comprises computer-executable instructions. The stored computer program code can include a program for natural-language processing that implements the disclosed methods. The data storage devices 111 may store the computer program code 107. Computer program code 107 stored in the storage devices 111 can be configured to be executed by processor 103 via the memory devices 105. Processor 103 can execute the stored computer program code 107.

Thus the present invention discloses a process for supporting computer infrastructure, integrating, hosting, maintaining, and deploying computer-readable code into the computer system 101, wherein the code in combination with the computer system 101 is capable of automatically and programmatically implementing the disclosed processes of the invention.

Any of the components of the present invention could be created, integrated, hosted, maintained, deployed, managed, serviced, supported, etc. by a service provider. Thus, the present invention discloses a process for deploying or integrating computing infrastructure, comprising integrating computer-readable code into the computer system 101, wherein the code in combination with the computer system 101 is capable of automatically and programmatically implementing the disclosed processes of the invention

One or more data storage units 111 (or one or more additional memory devices not shown in FIG. 3) may be used as a computer-readable hardware storage device having a computer-readable program embodied therein and/or having other data stored therein, wherein the computer-readable program comprises stored computer program code 107. Generally, a computer program product (or, alternatively, an article of manufacture) of computer system 101 may comprise said computer-readable hardware storage device.

While it is understood that program code 107 may be deployed by manually loading the program code 107 directly into client, server, and proxy computers (not shown) by loading the program code 107 into a computer-readable storage medium (e.g., computer data storage device 111), program code 107 may also be automatically or semi-automatically deployed into computer system 101 by sending program code 107 to a central server (e.g., computer system 101) or to a group of central servers. Program code 107 may then be downloaded into client computers (not shown) that will execute program code 107.

Alternatively, program code 107 may be sent directly to the client computer via e-mail. Program code 107 may then either be detached to a directory on the client computer or loaded into a directory on the client computer by an e-mail option that selects a program that detaches program code 107 into the directory.

Another alternative is to send program code 107 directly to a directory on the client computer hard drive. If proxy servers are configured, the process selects the proxy server code, determines on which computers to place the proxy servers' code, transmits the proxy server code, and then installs the proxy server code on the proxy computer. Program code 107 is then transmitted to the proxy server and stored on the proxy server.

In one embodiment, program code 107 is integrated into a client, server and network environment by providing for program code 107 to coexist with software applications (not shown), operating systems (not shown) and network operating systems software (not shown) and then installing program code 107 on the clients and servers in the environment where program code 107 will function.

The first step of the aforementioned integration of code included in program code 107 is to identify any software including the network operating system (not shown), which is required by program code 107 or that works in conjunction with program code 107 and is on the clients and servers where program code 107 will be deployed. This identified software includes the network operating system, where the network operating system comprises software that enhances a basic operating system by adding networking features. Next, the software applications and version numbers are identified and compared to a list of software applications and correct version numbers that have been tested to work with program code 107. A software application that is missing or that does not match a correct version number is upgraded to the correct version.

A program instruction that passes parameters from program code 107 to a software application is checked to ensure that the instruction's parameter list matches a parameter list required by the program code 107. Conversely, a parameter passed by the software application to program code 107 is checked to ensure that the parameter matches a parameter required by program code 107. The client and server operating systems, including the network operating systems, are identified and compared to a list of operating systems, version numbers, and network software programs that have been tested to work with program code 107. An operating system, version number, or network software program that does not match an entry of the list of tested operating systems and version numbers is upgraded to the listed level on the client computers and upgraded to the listed level on the server computers.

After ensuring that the software, where program code 107 is to be deployed, is at a correct version level that has been tested to work with program code 107, the integration is completed by installing program code 107 on the clients and servers.

Embodiments of the present invention may be implemented as a method performed by a processor of a computer system, as a computer program product, as a computer system, or as a processor-performed process or service for supporting computer infrastructure.

### Cloud Implementation

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

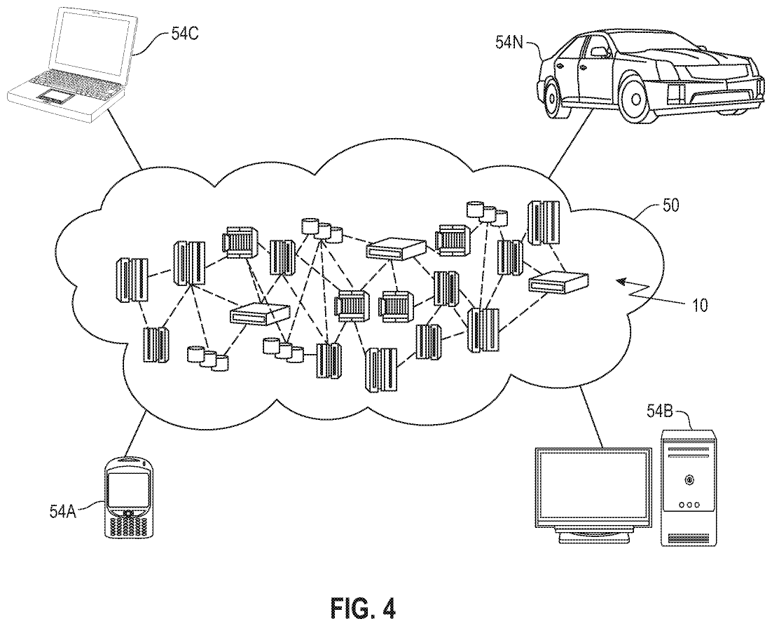

Referring now to FIG. 4, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that computing nodes 10 and cloud computing environment 50 can communicate with any type of computerized device over any type of network and/or network addressable connection (e.g., using a web browser).

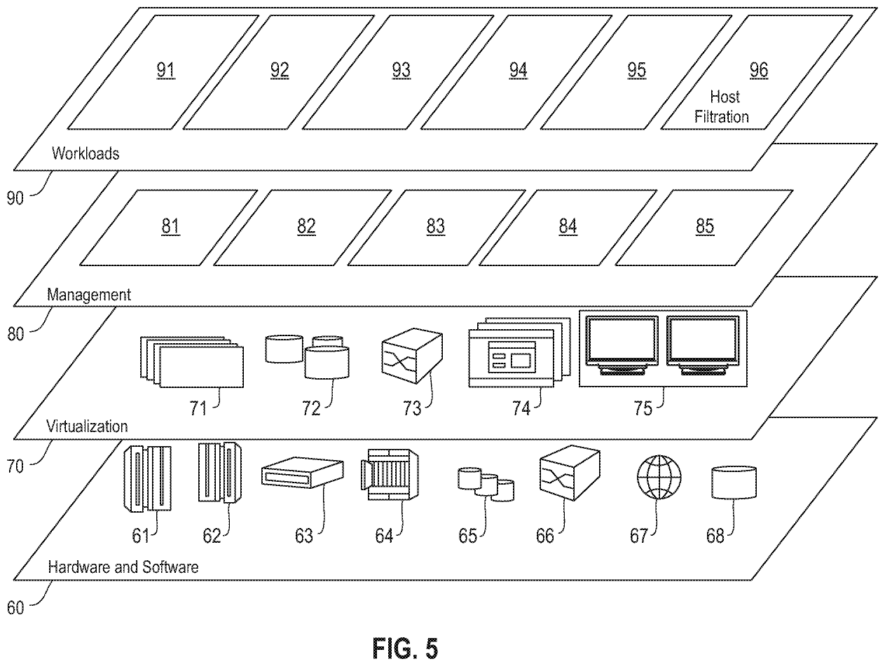

Referring now to FIG. 5, a set of functional abstraction layers provided by cloud computing environment 50 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and host sequence filtration 96.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. When a range is used to express a possible value using two numerical limits X and Y (e.g., a concentration of X ppm to Y ppm), unless otherwise stated the value can be X, Y, or any number between X and Y.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and their practical application, and to enable others of ordinary skill in the art to understand the invention.

What is claimed is:

1. A method, comprising:
obtaining an initial set of sequence reads from a sample containing DNA, RNA, and/or proteins of multiple organisms, the initial set comprising i) a first subset of sequences corresponding to a first organism and ii) a second subset of sequences corresponding to a second organism;
identifying the first organism;
forming an electronic filter containing sequences of DNA, RNA, and/or proteins of the identified first organism; and
removing from the initial set any sequences of DNA, RNA, and/or proteins matching the sequences of DNA, RNA, and/or proteins of the electronic filter, thereby forming a set of removed sequences and a set of remaining sequences;
wherein
the set of remaining sequences is suitable for identifying the second organism.

2. The method of claim 1, wherein said identifying the first organism is accomplished using between 0% and 20% of the sequence reads of the initial set.

3. The method of claim 1, wherein the second organism is a microorganism selected from the group consisting of bacteria, fungi, viruses, protozoans, and parasites.

4. The method of claim 1, wherein the sample is a food safety sample.

5. The method of claim 1, wherein the sample is a medical sample.

6. The method of claim 1, wherein the initial set is a product resulting from a quality control process performed on a raw set of sequences received directly from a sequencer device, the quality control process including one or more of i) purging sequences of poor quality, ii) removing contaminating sequences introduced by a sequencer device, and iii) removing sequences of low complexity.

7. The method of claim 1, wherein the method is performed by a cloud-based software service.

8. A computer program product, comprising a computer readable hardware storage device having a computer-readable program code stored therein, said program code configured to be executed by a processor of a computer system to implement a method comprising:
obtaining an initial set of sequence reads from a sample containing DNA, RNA, and/or proteins of multiple organisms, the initial set comprising i) a first subset of sequences corresponding to a first organism and ii) a second subset of sequences corresponding to a second organism;
identifying the first organism;
forming an electronic filter containing sequences of DNA, RNA, and/or proteins of the identified first organism; and
removing from the initial set any sequences of DNA, RNA, and/or proteins matching the sequences of DNA, RNA, and/or proteins of the electronic filter, thereby forming a set of removed sequences and a set of remaining sequences;
wherein
the set of remaining sequences is suitable for identifying the second organism.

9. The computer program product of claim 7, wherein the computer program product implements the method using multiple microservices configured for parallel processing.

10. The computer program product of claim 7, wherein the computer program product is performed by a cloud software service configured to perform a sequence filtration service.

11. A system comprising one or more computer processor circuits configured and arranged to:
obtain an initial set of sequence reads from a sample containing DNA, RNA, and/or proteins of multiple organisms, the initial set comprising i) a first subset of sequences corresponding to a first organism and ii) a second subset of sequences corresponding to a second organism;
identify the first organism;
form an electronic filter containing sequences of DNA, RNA, and/or proteins of the identified first organism; and
remove from the initial set any sequences of DNA, RNA, and/or proteins matching the sequences of DNA, RNA, and/or proteins of the electronic filter, thereby forming a set of removed sequences and a set of remaining sequences;
wherein
the set of remaining sequences is suitable for identifying the second organism.

12. The system of claim 11, wherein the system comprises multiple microservices configured for parallel processing of one or more data streams of the sequence reads of the initial set.

13. The system of claim 11, wherein the system is a component of a cloud-based software service.

14. The system of claim 13, wherein the cloud-based software service maintains a collection of electronic filters of previous removed sequences, and the sequence reads of the initial set are filtered through each electronic filter of the collection.

15. The system of claim 13, wherein the cloud-based software service receives raw sequence reads as a data stream uploaded directly from a nucleic acid sequencer device.

16. The system of claim 15, wherein the raw sequence reads from the sequencing device are processed through a quality control microservice of the system, thereby producing the initial set and a second set comprising failed reads, the initial set passed to other microservices of the system as one or more data streams, the second set stored in a failed read repository.

17. The system of claim 16, wherein said identify the first organism begins before the quality control microservice completely processes all of the raw sequence reads.

18. The system of claim 16, wherein an identification microservice of the system begins said identify the first organism before all of the raw sequence reads have been uploaded from the sequencer device.

19. The system of claim 16, wherein a filter microservice begins forming the electronic filter before all of the raw sequence reads have been uploaded from the sequencer device.

20. The system of claim 16, wherein a filter microservice begins forming the electronic filter before all of the raw sequences have been processed by the quality control microservice.

21. The system of claim 11, wherein the cloud-based software service maintains a repository of the removed sequences separate from a repository of the remaining sequences.

22. The system of claim 21, wherein all of the sequences previously stored in the repository of the remaining sequences are filtered through the electronic filter, and any removed sequences therefrom are moved to the repository of removed sequences.

23. A method, comprising:
inputting a sample into a sequencer, the sample including both a microbial target and a matrix on which the microbial target exists, the sequencer outputting reads of nucleic acids detected in the sample;
subjecting the reads to a quality control process to eliminate (i) part or all of those reads below a desired threshold and/or (ii) those reads originating from contaminants unrelated to the target or matrix;
determining the matrix identity by string matching a minority of the reads;
retrieving sequences related to the determined matrix by accessing a reference database;
in view of the retrieved sequences, dividing the reads into a first object store directed to the matrix and a second object store directed to the microbial target and any residuals, thus effectively filtering the host from the microbial target; and
using at least one of the object stores in a follow-on application.

24. The method of claim 22, wherein the follow-on application is pathogen tracking.

25. The method of claim 22, wherein the follow-on application measures antimicrobial resistance.

26. The method of claim 22, wherein the first object store is used in the follow-on application.

27. The method of claim 22, wherein the second object store is used in the follow-on application.

28. The method of claim 22, wherein said determining the matrix identity is performed with no a priori knowledge of the matrix identity.

29. The method of claim 22, wherein the matrix identity is procured dynamically for a given sample and a filter is generated dynamically based on that identity, the filter being used to divide reads as (i) matrix and (ii) microbial target with any residuals.
